(19) United States
(12) Patent Application Publication
(10) Pub. No.: US 2026/0022369 A1
(43) Pub. Date: Jan. 22, 2026

(54) COMPOSITIONS AND METHODS FOR DETERMINING GENOME ORGANIZATION AT HIGH SPATIAL AND GENOMIC RESOLUTION

(71) Applicants: Board of Regents, The University of Texas System, Austin, TX (US); Baylor College of Medicine, Houston, TX (US)

(72) Inventors: Guy Nir, Galveston, TX (US); Kadir Akdemir, Houston, TX (US); Erez Liberman Aiden, Houston, TX (US)

(21) Appl. No.: 19/271,152

(22) Filed: Jul. 16, 2025

Related U.S. Application Data
(60) Provisional application No. 63/673,339, filed on Jul. 19, 2024.

(51) Int. Cl. C12N 15/10; C12Q 1/6806; C12Q 1/6816; C12Q 1/6844

(52) U.S. Cl. CPC C12N 15/1065; C12Q 1/6806; C12Q 1/6816; C12Q 1/6844

(57) ABSTRACT

Provided herein are systems, methods, and kits for determining genome organization at high spatial and genomic resolution comprising: obtaining or having obtained one or more chromosomes; binding to the one or more chromosomes a library comprising one or more imaging oligonucleotides, comprising a genome homology region sequence that binds to a genomic sequence, two or more barcode sequences, and two or more universal primer sequences; imaging the one or more chromosomes at a first resolution by subdividing the chromosome into two or more first segments having a first length; imaging the two or more first segments at a second resolution by subdividing each of the two or more first segments into two or more second segments having a second length; and subsequently imaging additional subsegments by subdividing each prior segment into two or more subsequent segments having two or more smaller lengths.

### CROSS-REFERENCE TO RELATED APPLICATIONS

This application claims priority to U.S. Provisional Application Ser. No. 63/673,339, filed Jul. 19, 2024 entitled “Compositions and Methods for Determining Genome Organization at High Spatial and Genomic Resolution”, which is hereby incorporated by reference in its entirety.

### TECHNICAL FIELD

The present invention relates in general to the field of genome organization at high spatial and genomic resolution, and more particularly, to novel compositions and methods for determining genome organization at high spatial and genomic resolution.

### STATEMENT OF FEDERALLY FUNDED RESEARCH

None.

### INCORPORATION-BY-REFERENCE OF MATERIALS FILED ON COMPACT DISC

Not applicable.

### BACKGROUND

Without limiting the scope of the invention, its background is described in connection with methods for determining genome organization.

In recent years, the field of genome organization has benefited much by using sequential imaging approaches to trace chromosomal segments at the single cell level. Thus, allowing to identify tens, hundreds, and even thousands of loci from the same cells. However, these microscopy approaches only allow a few tens of targets to be identified in the same cells and are subjected to diffraction-limited resolution. This limits their capacity to describe the structure of nanoscale chromatin structures.

At the other end, super-resolution chromatin tracing achieves nanoscale resolution through Single Molecule Localization Microscopy (SMLM), such as, Stochastic Optical Reconstruction Microscopy (STORM). However, STORM is considered a ‘slow’ technology, and thus, sequentially imaging many genomic targets to resolve genome organization in high genomic resolution is extremely challenging.

However, despite these advances, a need remains for novel compositions and methods for determining genome organization at high spatial and genomic resolution.

### SUMMARY OF THE INVENTION

As embodied and broadly described herein, an aspect of the present disclosure relates to a system of determining genome organization at high spatial and genomic resolution comprising: a microscope for imaging one or more chromosomes fixed on a substrate; a library comprising one or more imaging oligonucleotides capable of binding to the one or more chromosomes, each imaging oligonucleotide comprising a genome homology region sequence that binds to a genomic sequence, two or more barcode sequences, and two or more universal primer sequences; reagents for amplification of two or more barcodes with universal primers; wherein the microscope is capable of capturing: one or more images of the one or more chromosomes at a first resolution by subdividing the one or more chromosome into two or more first segments having a first length; imaging the two or more first segments at a second resolution by subdividing each of the two or more first segments into two or more second segments having a second length; and subsequently imaging additional subsegments by subdividing each prior segment into two or more subsequent segments having two or more smaller lengths; and a processor capable of processing the one or more images at a first, second or subsequent resolutions, to determine a resolution of nested images between the lowest and highest resolutions. In one aspect, the barcode sequences are 3′, 5′, or both 3′ and 5′ from the genome homology region sequence. In another aspect, the system further comprises increasing or decreasing the lengths of the one or more subsequent segments to increase one or more times a resolution of the genome organization at high spatial and genomic resolution. In another aspect, the barcode sequences are amplified with a universal primer and a chromophore or visualizing agent. In another aspect, the first segment has a length of an entire chromosome, the second segment subdivides the length of the first segment 2, 3, 4, 5, 6, 7, 8, 9, 10, 15, 20, 25, 30, 35, 40, 45, 50, 60, 70, 80, 90, 100, 200, 250, 300, 400, 500, 600, 700, 750, 800, 1,000, 2,000, 3,000, 4,000, 5,000, 6,000, 7,000, 8,000, 9,000, 10,000 times or more, and wherein each additional subdivision of additional subsegments is subdivided 2, 3, 4, 5, 6, 7, 8, 9, 10, 15, 20, 25, 30, 35, 40, 45, 50, 60, 70, 80, 90, 100 times or more, down to a resolution of an oligonucleotide. In another aspect, the genome organization imaged is used to determine one or more of the following: loop domain borders, structural differences between chromosome compartments and homologous chromosomes, how loop stacking organizes chromatin folding, and how gene activity is directly linked to chromosome structure. In another aspect, the imaging is by Single Molecule Localization Microscopy (SMLM) or Stochastic Optical Reconstruction Microscopy (STORM). In another aspect, the chromosomes are traced by ball-and-stick tracing (BST) or volumetric chromatin tracing (VCT). In another aspect, the imaging additional subsegments by subdividing each prior segment into two or more subsequent segments is selected from 1, 2, 3, 4, 5, 6, 7, 8, 9, 10, 11, 12, 13, 15, 16, 17, 18, 19, 20, 25, 30, 40, 50 or more subsequent rounds or waves of subdivisions. In another aspect, the subdivision of segments follows the formula: y=x\hat{ }n, where y is the number of total targets (imaged loci), x is the number of rounds of imaging per wave, and n is the number of waves. In another aspect, the imaging of the one or more chromosomes achieves a resolution of 8, 10, 20, 30, 40, 50, 60, 70, 80, 90, 100, 200, 250, 300, 400, 500, 750, 1,000 bp, 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 kb, or 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 Mb, or portions of a chromosome. In another aspect, the one or more chromosomes are imaged concurrently.

As embodied and broadly described herein, an aspect of the present disclosure relates to a method of determining genome organization at high spatial and genomic resolution comprising: obtaining or having obtained one or more chromosomes; binding to the one or more chromosomes a library comprising one or more imaging oligonucleotides, each imaging oligonucleotide comprising a genome homology region sequence that binds to a genomic sequence, two or more barcode sequences, and two or more universal primer sequences; imaging the one or more chromosomes at a first resolution by subdividing the one or more chromosome into two or more first segments having a first length; imaging the two or more first segments at a second resolution by subdividing each of the two or more first segments into two or more second segments having a second length; and subsequently imaging additional subsegments by subdividing each prior segment into two or more subsequent segments having two or more smaller lengths. In one aspect, the barcode sequences are 3′, 5′, or both 3′ and 5′ from the genome homology region sequence. In another aspect, the barcode sequences are amplified with a universal primer and a chromophore or visualizing agent. In another aspect, the method further comprises increasing or decreasing the lengths of the one or more subsequent segments to increase one or more times a resolution of the genome organization at high spatial and genomic resolution. In another aspect, the first segment has a length of an entire chromosome, the second segment subdivides the length of the first segment 2, 3, 4, 5, 6, 7, 8, 9, 10, 15, 20, 25, 30, 35, 40, 45, 50, 60, 70, 80, 90, 100, 200, 250, 300, 400, 500, 600, 700, 750, 800, 1,000, 2,000, 3,000, 4,000, 5,000, 6,000, 7,000, 8,000, 9,000, 10,000 times or more, and wherein each additional subdivision of additional subsegments is subdivided 2, 3, 4, 5, 6, 7, 8, 9, 10, 15, 20, 25, 30, 35, 40, 45, 50, 60, 70, 80, 90, 100 times or more, down to a resolution of an oligonucleotide. In another aspect, the genome organization imaged is used to determine one or more of the following: loop domain borders, structural differences between chromosome compartments and homologous chromosomes, how loop stacking organizes chromatin folding, and how gene activity is directly linked to chromosome structure. In another aspect, the imaging is by Single Molecule Localization Microscopy (SMLM) or Stochastic Optical Reconstruction Microscopy (STORM). In another aspect, the chromosomes are traced by ball-and-stick tracing (BST) or volumetric chromatin tracing (VCT). In another aspect, the imaging additional subsegments by subdividing each prior segment into two or more subsequent segments is selected from 1, 2, 3, 4, 5, 6, 7, 8, 9, 10, 11, 12, 13, 15, 16, 17, 18, 19, 20, 25, 30, 40, 50 or more subsequent rounds or waves of subdivisions. In another aspect, the subdivision of segments follows the formula: y=x\hat{ }n, where y is the number of total targets (imaged loci), x is the number of rounds of imaging per wave, and n is the number of waves. In another aspect, the imaging of the one or more chromosomes achieves a resolution of 8, 10, 20, 30, 40, 50, 60, 70, 80, 90, 100, 200, 250, 300, 400, 500, 750, 1,000 bp, 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 kb, or 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 Mb, or portions of a chromosome. In another aspect, the one or more chromosomes are imaged concurrently.

As embodied and broadly described herein, an aspect of the present disclosure relates to a kit comprising in one or more vials: one or more reagents for fixing one or more chromosomes; a library capable of binding to the one or more chromosomes comprising one or more imaging oligonucleotides, wherein each imaging oligonucleotide comprising a genome homology region sequence that binds to a genomic sequence, two or more barcode sequences, and two or more universal primer sequences; one or more universal oligonucleotides; one or more amplification reagents for amplification of products from the two or more barcode sequences; one or more imaging reagents for imaging the amplification products from the two or more barcode sequences; and instructions for use of the kit to image and determine a genome organization at high spatial and genomic resolution. In one aspect, the barcode sequences are 3′, 5′, or both 3′ and 5′ from the genome homology region sequence.

In another aspect, the barcode sequences are amplified with a universal primer and a chromophore or visualizing agent. In another aspect, the first segment has a length of an entire chromosome, the second segment subdivides the length of the first segment 2, 3, 4, 5, 6, 7, 8, 9, 10, 15, 20, 25, 30, 35, 40, 45, 50, 60, 70, 80, 90, 100 times or more, and wherein each additional subdivision of additional subsegments is subdivided 2, 3, 4, 5, 6, 7, 8, 9, 10, 15, 20, 25, 30, 35, 40, 45, 50, 60, 70, 80, 90, 100, 200, 250, 300, 400, 500, 600, 700, 750, 800, 1,000, 2,000, 3,000, 4,000, 5,000, 6,000, 7,000, 8,000, 9,000, 10,000 times or more, down to a resolution of an oligonucleotide. In another aspect, the genome organization imaged is used to determine one or more of the following: loop domain borders, structural differences between chromosome compartments and homologous chromosomes, how loop stacking organizes chromatin folding, and how gene activity is directly linked to chromosome structure. In another aspect, the imaging is by Single Molecule Localization Microscopy (SMLM) or Stochastic Optical Reconstruction Microscopy (STORM). In another aspect, the chromosomes are traced by ball-and-stick tracing (BST) or volumetric chromatin tracing (VCT). In another aspect, the imaging additional subsegments by subdividing each prior segment into two or more subsequent segments is selected from 1, 2, 3, 4, 5, 6, 7, 8, 9, 10, 11, 12, 13, 15, 16, 17, 18, 19, 20, 25, 30, 40, 50 or more subsequent rounds or waves of subdivisions. In another aspect, the subdivision of segments follows the formula: y=x\hat{ }n, where y is the number of total targets (imaged loci), x is the number of rounds of imaging per wave, and n is the number of waves. In another aspect, the imaging of the one or more chromosomes achieves a resolution of 8, 10, 20, 30, 40, 50, 60, 70, 80, 90, 100, 200, 250, 300, 400, 500, 750, 1,000 bp, 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 kb, or 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 Mb, or portions of a chromosome. In another aspect, the one or more chromosomes are imaged concurrently.

### BRIEF DESCRIPTION OF THE DRAWINGS

For a more complete understanding of the features and advantages of the present disclosure, reference is now made to the detailed description of the disclosure along with the accompanying figures and in which:

FIG. 1 is a schematic summary that provides a high level overview of the chromosome imaging of the present invention.

FIGS. 2A and 2B show the basic outline of the matryoshka method. FIG. 2A. Design of Oligopaint oligo compatible with Matryoshka. Multiple barcodes can be appended to the genome homology region. Those upstream of the genome homology region are referred to as mainstreets and those downstream of it are referred to as backstreets. FIG. 2B. The Matryoshka imaging outline. In the first wave (out of the four depicted in the figure), the largest intervals are imaged sequentially. Then, smaller intervals are imaged in parallel and are identified through the analysis.

FIGS. 3A to 3D show the design of Matryoshka libraries and preliminary images. FIG. 3A) Graphical representation of Matryoshka design for the EGFR library. The first 20 steps divide the region into larger intervals of 100 kb and the last 5 steps further subdivide each Mainstreet step to bring the genomic resolution to 20 kb. FIG. 3B) Graphical representation of Matryoshka design for the TwoChrs library. The first 25 steps divide the region into larger intervals of 500 kb and the last 5 steps further subdivide each Mainstreet step to bring the genomic resolution to 100 kb. FIG. 3C) Image of all 25 steps of the EGFR library. FIG. 3D) Image of all 30 steps of the TwoChrs library. Each sphere in C & D represents a single blink. Pseudo-colors represent rounds of imaging.

FIGS. 4A to 4D shows the backstreet assignment workflow. FIG. 4A, Matryoshka library design. FIG. 4B, Dimension reduction by finding the center-of-mass (blue) of data points (orange) in a sphere of radius, R. FIG. 4C, the shortest path between data center-of-mass points predicted by graph algorithms (red lines). FIG. 4D, Backstreet segments are assigned to their corresponding mainstreet steps by calculating the distance between each backstreet particle and graph edge.

FIGS. 5A to 5C show models performance against randomly assign sub-segments for chromosome 9 trace 1 analysis. FIG. 5A, fraction of BS localizations across different MS steps. FIG. 5B, Distributions for pairwise distances between BS particles and their assigned edges for predictions R=100 nm, R=200 nm, and random sub-segment assignment. FIG. 5C, box plot comparison for predictions R=100 nm, R=200 nm, and random sub-segment assignment.

FIGS. 6A TO 6E: Center-to-center contact maps for chromosome 9 trace 1 analysis. FIG. 6A, center-to-center pairwise distances between MS time points. FIG. 6B, center-to-center pairwise distances between BS time points after their assignment in course/low resolution binning. FIG. 6C, center-to-center pairwise distances between BS time points after their assignment in fine/high resolution binning. FIG. 6D, center-to-center pairwise distances between BS time points after their random assignments in course/low resolution binning. FIG. 6E, center-to-center pairwise distances between BS time points after their random assignments in fine/high resolution binning.

FIGS. 7A and 7B show the results from using Matryoshka. FIG. 7A shows the the mean center-to-center distance matrix, compared to FIG. 7B using Hi-C, which is a well-known method for 3D genome organization at the ensemble level). The graphs are at 20 kb, using Matryoshka from 100 kb to 20 kb.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

The present inventors have developed novel compositions and methods for determining genome organization at high spatial and genomic resolution. Nicknamed, Matryoshka, after Russian nesting dolls, the novel compositions and methods overcome the limitations of the prior art through a sophisticated approach of labeling and imaging oligos, followed by a computational analysis to identify the genomic position of each genomic target. This type of analysis relies on the output of Single Molecule Localization Microscopy (SMLM), such as, Stochastic Optical Reconstruction Microscopy (STORM), and thus is unique to super-resolution tracing. A benefit of Matryoshka, is that it does not require adding many rounds of imaging to identify thousands of targets, and is therefore more efficient than any other known tracing technology. Using the present inventors' novel compositions and methods, the entire human genome can be imaged with 40 rounds of imaging achieving a genomic resolution of 5-25 kb (depending on the size chromosome) and with nanoscale resolution.

As used herein, the term “barcode sequence” refers to a unique nucleotide sequence for a corresponding nucleic acid base and/or nucleic acid sequence to be identified. Often, barcode sequences can each have a length of from about 5 nucleotides to about 40 nucleotides. For example, barcode sequences can each have a length of from about 5 nucleotides to about 35 nucleotides, from about 6 nucleotides to about 30 nucleotides, from about 7 nucleotides to about 25 nucleotides, from about 8 nucleotides to about 15 nucleotides. In some embodiments, barcode sequences can each have a length of 5, 6, 7, 8, 9, 11, 12, 13, 14, 15, 16, 17, 18, 19, 20, 21, 22, 23, 24, 25, 26, 27, 28, 29, 30, 31, 32, 33, 34, 34 or 35 nucleotides.

As used herein, the term “detectable label” refers a moiety capable of producing a detectable signal. The barcode sequence, or other sequences for use with the present invention, will often be paired with a specific detectable label. Detectable labels can include any molecule, composition, or combination of molecules that are detectable by spectroscopic, photochemical, biochemical, immunochemical, electrical, electromagnetic, optical, chemical, mechanical and the like. Detectable labels can include light-absorbing dyes, fluorescent molecules, radioisotopes, nucleotide chromophores, enzymes, substrates, chemiluminescent, bioluminescent, mass labels, electron dense particles, magnetic particles, spin labels, charged groups, nanoparticles, quantum dots, and the like. The detectable labels used in the methods described herein can be primary labels (where the label comprises a molecule or moiety that is directly detectable or that produces a directly detectable molecule or moiety) or secondary labels (where the detectable label binds to another molecule or moiety to produce a detectable signal, e.g., as is common in immunological labeling using secondary and tertiary antibodies). In some embodiments, the detectable label can include biotin, amines, metals, metal nanoclusters (e.g., gold, silver, platinum, or copper), metal nanoparticles (e.g., gold, silver, platinum, or copper), anchoring molecules, quantum dots, fluorescent polydots or acrydite.

Fluorophores for use with the present invention includes any that can be attached to the one or more probes taught herein. Fluorescent molecule or moiety emits light energy at a specific emission wavelength when excited by an appropriate excitation wavelength. Exemplary fluorophores include, e.g., 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Acquorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; JF 549, JF 646, JF 594, CF 488, CF 568, CF 647, CF 750, CF 660, SIR, HMSIR, Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calccin; Calcein Blue; Calcium Crimson™; Calcium Green; Calcium Green-1 Ca.sup.2+ Dye; Calcium Green-2 Ca.sup.2+; Calcium Green-5N Ca.sup.2+; Calcium Green-C18 Ca.sup.2+; Calcium Orange; Calcofluor White; Carboxy-X-rhodamine (5-ROX); Cascade Blue™; Cascade Yellow; Catecholamine; CFDA; CFP-Cyan Fluorescent Protein; Chlorophyll; Chromomycin A; Chromomycin A; CMFDA; Coclenterazine; Coclenterazine cp; Coclenterazine f; Coclenterazine fcp; Coclenterazine h; Coclenterazine hep; Coelenterazine ip; Coclenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodaminc 123); Di-4-ANEPPS; Di-8-ANEPPS (non-ratio); DiA (4-Di-16-ASP); DIDS; Dihydorhodamine 123 (DHR); DiO (DiOC18 (3)); DiR; DIR (DiIC18 (7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosanilinc); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Hacmatoporphyrin; Hoechst 33258; 33342; Hoechst Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissaminc Rhodaminc B; LOLO-1; LO-PRO-1; Lucifer Yellow; Mag Green; Magdala Red (Phloxin B); Magnesium Green; Magnesium Orange; Malachite Green; Marina Bluc; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimanc (mBBr-GSH); Monochlorobimane; MPS (Methyl Green Pyroninc Stilbene); NBD; NBD Amine; Nile Red; Nitrobenzoxadidole; Noradrenaline; Nuclear Fast Red; Nuclear Yellow; Nylosan Brilliant lavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycocrythrin B [PE]; Phycocrythrin R [PE]; PKH26; PKH67; PMIA; Pontochrome Blue Black; POPO-1; POPO-3; PO-PRO-1; PO-PRO-3; Primuline; Procion Yellow; Propidium Iodide (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocrythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66 W; Yellow GFP; YFP; YO-PRO-1; YO-PRO-3; YOYO-1; and YOYO-3. Many suitable forms of these fluorescent compounds are available and can be used. In some embodiments, a combination of different fluorophores is used, for example, to distinguish between A, T, C, or G nucleotides (i.e., sequential fluorophores) and to reduce background noise.

Certain embodiments of the present invention may include one or more steps in which a target nucleic acid strand is amplified. These embodiments can use any of the many methods for amplifying nucleic acid sequences, e.g., isothermal amplification, polymerase chain reaction (PCR) and variants of PCR such as multiplex RT-PCR, immuno-PCR, SSIPA, Real Time RT-qPCR and nanofluidic digital PCR. In some embodiments, the docking strand is amplified using an isothermal amplification. Non-limiting examples of isothermal amplification include, but are not limited to, Recombinase Polymerase Amplification (RPA), nested RPA, Loop Mediated Isothermal Amplification (LAMP), Helicase-dependent isothermal DNA amplification (HDA), thermophilic helicase-dependent amplification (tHDA), Nucleic acid sequence-based amplification (NASBA), strand displacement amplification (SDA), ligase chain reaction (LCR), nicking enzyme amplification reaction (NEAR), Polymerase Spiral Reaction (PSR), polymerase cross-linking spiral reaction (PCLSR), and transcription-based amplification systems (TAS) such as nucleic acid sequence based amplification (NASBA), Rolling Circle Amplification (RCA), and Rapid Amplification of cDNA Ends (RACE, “one-sided PCR”). Non-isothermal amplification methods can also be used e.g., PCR, multiple displacement amplification (MDA), rolling circle amplification (RCA), ligase chain reaction (LCR), or ramification amplification method (RAM).

Embodiments of the present invention may workchr as follows. A chromatin segment, for example, the largest human chromosome, chromosome 1 (250 Mb), can be divided into ten segments, of 25 Mb. Those will be imaged sequentially in ten rounds of imaging. This is called the first Matryoshka ‘wave’ or ‘round’.

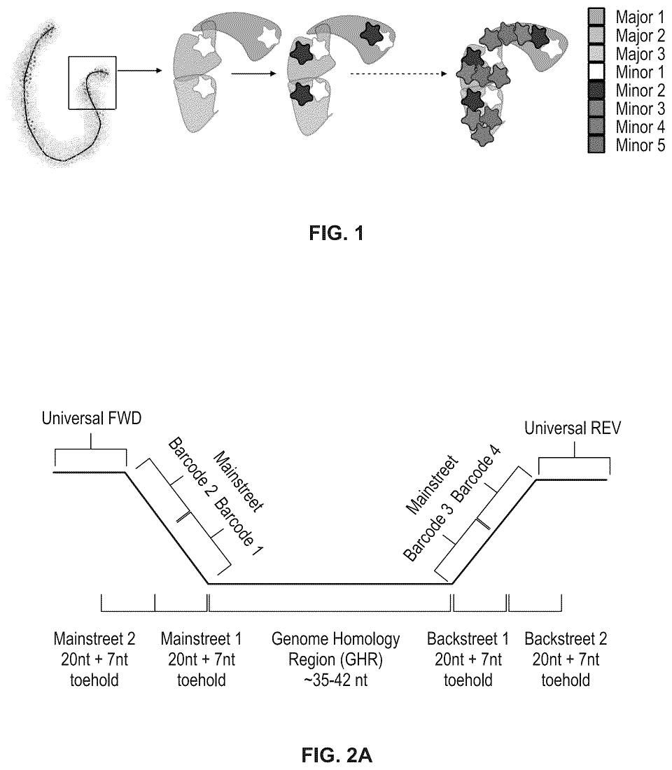

The unique design of a Matryoshka oligo library includes several barcodes on each oligo, which further segment each larger segment. In the second Matryoshka wave, each 25 Mb segment is divided into ten more segments (of 2.5 Mb each), and the first segment out of the ten is imaged simultaneously for all 25 Mb segments (see FIG. 1). In the next round of imaging (round 12), the second segment of each 25 Mb segments will be imaged. After 20 rounds of imaging, chromosome I will be imaged at 2.5 Mb resolution. Since the 2.5 Mb segments are imaged simultaneously, these will be mapped to their corresponding larger segment, that were imaged sequentially in the first Matryoshka wave.

A person of skill in the art would readily recognize that steps of various above-described methods can be performed by one or more programmed computers, each having one or more computer processors. Herein, some embodiments are also intended to cover program storage devices, e.g., digital data storage media, which are machine or computer-readable and encode machine-executable or computer-executable programs of instructions, wherein the instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

The computational analysis relies on matching localizations (centroid of each detected fluorophore with STORM) from previous waves with current waves.

Now each 2.5 Mb is further segmented to ten 250 kb segments, and these will be further segmented into ten 25 kb segments. Thus, with 40 rounds of imaging (4 Matryoshka waves), the entire chromosome 1 will be imaged at 25 kb resolution, which is 10,000 targets. This stands in stark contrast to sequentially imaging 40 targets, which would result in 250/40=6.25 Mb resolution. Thus, Matryoshka improves exponentially the efficiency of identified targets and therefore, the genomic resolution. Four Matryoshka waves with ten rounds of imaging in each can be described with this equation-4 \hat{ }10=10,000 (and therefore, exponential improvement).

Since different chromosomes do not tend to entangle much, those chromosomes may be imaged simultaneously, therefore, with 40 rounds of imaging it was possible to resolve the entire human genome with 25 kb resolution. Thus, Matryoshka, improves the ability to determine genome organization at the single-cell level with nanoscale resolution better than any other known technique.

Single-cell technologies for studying genome organization have uncovered valuable information such as the cell-to-cell variability in loop domain borders¹, the structural differences between chromosome compartments²⁻⁵ and homologous chromosomes^{3,6}, how loop stacking organizes chromatin folding⁷, and how gene activity is directly linked to chromosome structure⁸⁻¹⁰. These single-cell approaches can largely be divided into two categories, those that rely on sequencing¹¹⁻¹⁶ and those who lean on imaging¹⁷⁻¹⁹. While sequencing-based approaches tend to be genome-wide and offer a high-genomic resolution, microscopy-based approaches visualize chromatin interactions inside the cells, and are often expanded to include RNA, proteins, and nuclear bodies^{4, 9, 20, 21}.

Among the microscopy-based approaches, chromatin tracing is perceived as the technology which can describe the structure of many loci within the same cells. Chromatin tracing, by itself, can be divided into two categories, which are based on the microscopes used^{17, 22}. The first relies on diffraction-limited microscopes and is often referred to as Ball-and-Stick tracing (BST). The second category refers to Volumetric chromatin tracing (VCT), which uses super-resolution microscopes. Among these super-resolution microscopy approaches, Single Molecule Localization Microscopy (SMLM) allows for the detection of single fluorophores. That is, SMLM-driven VCT (smVCT) records the position of each oligo bound to the chromatin. This contrasts with BST, which considers all oligos bound to a chromosomal segment as one and averages over their signal to find the centroid. In this way, BST loses valuable structural information and relies on modeling, at best, to connect the dots (centroids). In addition, BST suffers from optical crowding²³. That is, when many readout probes (dye-conjugated oligos) are excited and fluoresce simultaneously, their point spread functions (PSFs) overlap and cannot be resolved. This puts a limit on the number of targets that can be detected simultaneously. For example, Takei et al used 80 rounds of imaging to detect 3,660 loci²⁴. Recently, the same group published a preprint which shows a great improvement, which enables ˜100,000 loci in 96 rounds of imaging⁴. So, despite the optical crowding, current BST approaches are superior to smVCT, which has been so far executed in a mostly sequential manner.

Not only has smVCT so far been limited in its multiplexity, but sit is also slow, which is an intrinsic flaw of SMLM that requires the recording of many frames to eventually reconstruct an image. This puts a practical limit on the number of targets that can be visualized within a single cell using smVCT. For instance, it can take up to 10 days to sequentially visualize 36 targets in 10-20 IMR90 (human fibroblast) cells. To compare, with BST, on average, ˜2,000 cells may take 3 days for imaging ˜1,000 targets with 50 rounds of imaging⁹.

The inventors sought to develop a technology that is capable of imaging many genomic targets in a highly efficient way using SMLM. It was reasoned that if the method sequentially images a chromosomal region with N rounds of imaging, the imaged regions can then be divided into into N sub-segments and image each of the N sub-segments in parallel in N rounds of imaging. That is, 2N rounds of imaging will visualize N² targets, which means that the number of repeated rounds of imaging, X, becomes the exponent: X*N=N^{X}. As an example, the inventors used four rounds of repeated imaging, which are referred to as ‘waves’, with ten rounds of imaging in each wave. The number of rounds can be adjusted up and down based on the resolution sought, e.g., the invention can use 1, 2,3, 4, 5, 6, 7, 8, 9, 10, 11, 12, 13, 14, 15, 16, 17, 18, 19, 20, or 25 rounds. This yields 4*10=40 targets in a typical smVCT, but will result in 10⁴ imaged targets using this approach. That is, in ˜11 days, instead of imaging 40 loci, this strategy can resolve 10⁴ loci.

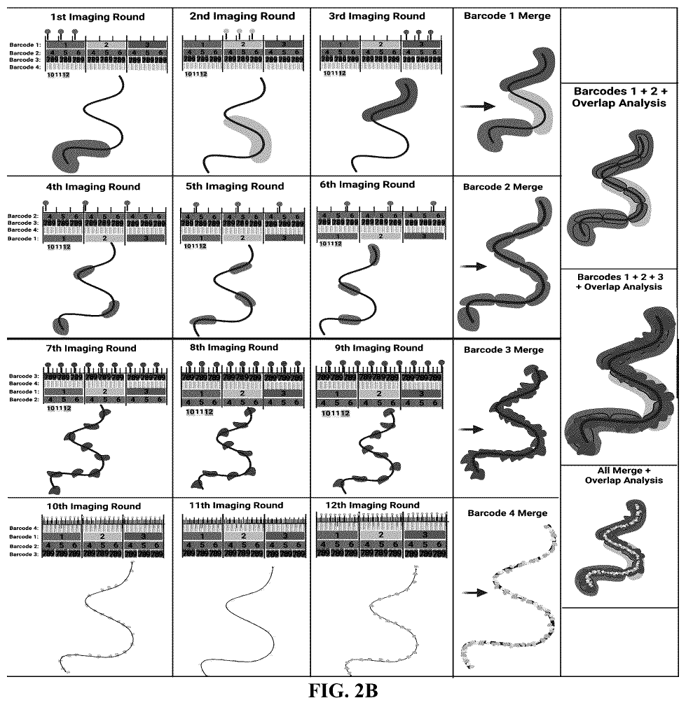

This technology requires re-imaging of the same segments but at smaller genomic intervals and in parallel, using Oligopaint oligos (FIG. 2A). The central piece of Oligopaint oligos is the genome homology region, which is flanked by a mainstreet (upstream) and a backstreet (downstream). Each street can be divided into several barcodes and a universal primer for library amplification³. Having multiple barcodes on the same oligo enables imaging each oligo several times. Here, a given barcode, e.g., ‘Barcode 1’, can be used to label a large interval. Barcode 2 will label a smaller interval (sub-interval) within the larger interval, whereas barcode 3 will label a smaller interval than the one imaged with barcode 2 (FIG. 2B).

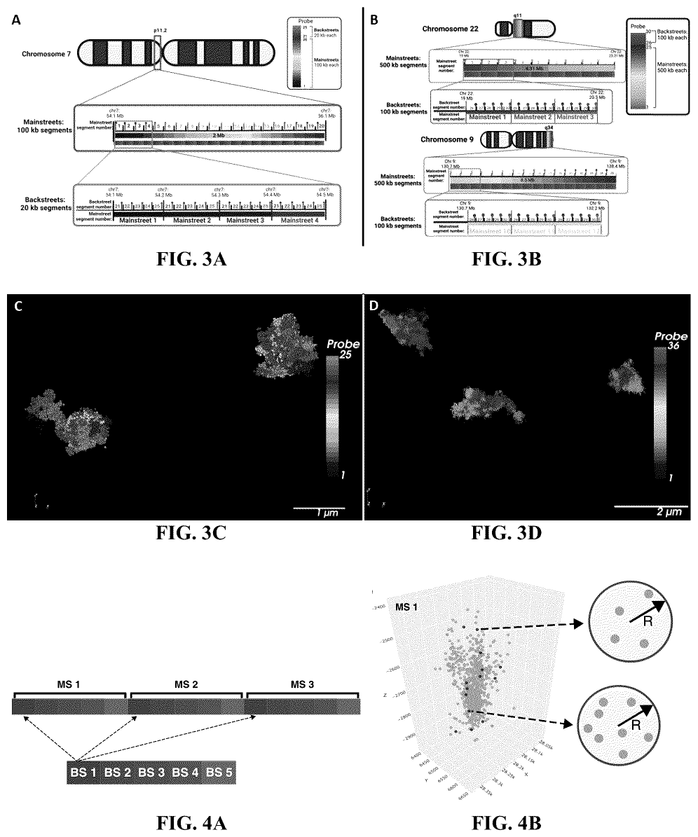

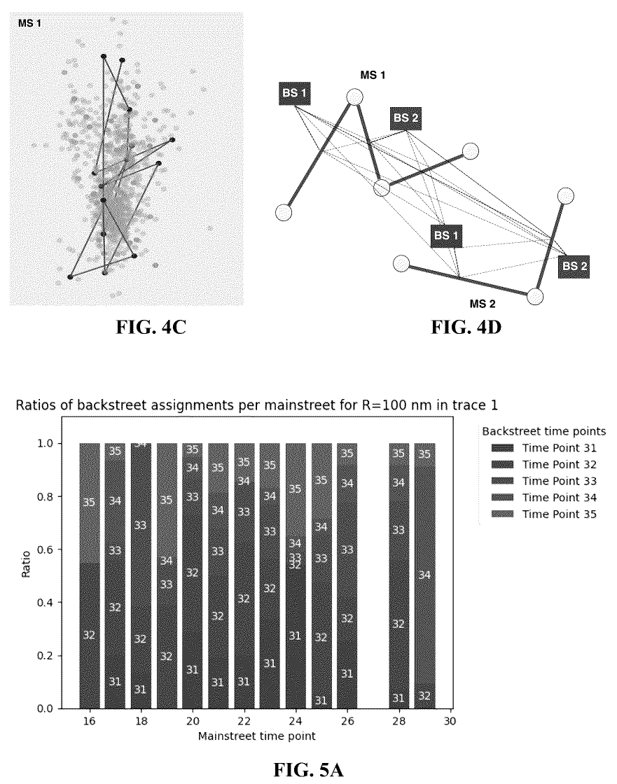

This technique, called ‘Matryoshka’ (nesting dolls in Russian, similarly to the nested segments), requires that the first wave will be imaged sequentially, to properly identify each larger segment. Then, in the next wave, the first sub-segments of each larger segment are imaged simultaneously. In round two of wave two, the second sub-segments of each larger segment will be imaged together, until all sub-segments of that wave have been imaged (FIGS. 2, 3A, & 3B). To assign the sub-segments to their corresponding larger segment an overlap analysis was performed (FIGS. 4A to 4D). This type of analysis requires data acquired with SMLM that produces a volumetric structure. Therefore, this approach is more appropriate to smVCT and makes this type of tracing highly efficient.

FIGS. 2A and 2B. show the basic matryoshka strategy. FIG. 2A. Design of Oligopaint oligo compatible with Matryoshka. Multiple barcodes can be appended to the genome homology region. Those upstream of the genome homology region are referred to as mainstreets and those downstream of it are referred to as backstreets. FIG. 2B. The matryoshka imaging outline, in the first wave (out of the four depicted in the figure), the largest intervals are imaged sequentially. Then, smaller intervals are imaged in parallel and are identified through the analysis summarized in FIGS. 4A to 4D.

Library design. To test Matryoshka for the first time, the inventors designed two Oligopaint libraries to be traced in two different human (hg38) cell types. The first library, named the ‘EGFR’ library, was imaged in microglial cells. This library is designed to target chr7: 54, 100,001-56,100,000, centered around the EGFR gene, and requires 25 rounds of imaging (FIG. 3A). The first 20 steps are done sequentially at 100 kb per step, and the last 5 steps are done in parallel, thus covering the entire 2 Mb at a resolution of 20 kb (5 steps*20 kb=100 kb). The second library, which was called the ‘TwoChrs’ library, targets segments of two chromosomes and was imaged in human fibroblasts (IMR90) cells (FIG. 3B). Chr9: 130,713,016-138,394,717 and Chr22: 19,000,000-23,318,037. Which is, ˜8 Mb of chromosome 9 and ˜4.3 Mb of chromosome 22. This library requires 30 rounds of imaging. The first 25 steps are imaged at 500 kb intervals, and the last five improve the resolution to 100 kb. The skilled artisan will recognize that all parts of any sequenced genome could be imaged with this technology. It is not limited to any organism or genetic element. Any genome or chromoscome can be imaged with the present invention. Non-limiting examples of genome that can be imaged with the present invention includes viral, bacteria, and/or eukaryotic genomes.

The inventors traced a total of X1 chromosomes in YI microglia cells of the EGFR library with an average lateral precision of A and axial precision of B nm. A total of X2 chromosomes in Y2 IMR90 cells of the TwoChrs library was traced with an average lateral precision of A1 and axial precision of B1 nm. FIGS. 3C and 3D provide a representative image of the EGFR and TwoChrs Oligopaints libraries (respectively) where each particle represents the centroid of a momentarily fluorescing (‘blinking’) A647N (Alexa Fluor 647 NHS Ester) dye and each pseudocolor corresponds to the round of imaging. Other examples can include visual comparisons between the traces imaged first through the mainstreets and then re-imaged through the backstreets.

FIGS. 3A to 3D show the design of matryoshka libraries and preliminary images. FIG. 3A) Graphical representation of Matryoshka design for the EGFR library. The first 20 steps divide the region into larger intervals of 100 kb and the last 5 steps further subdivide each Mainstreet step to bring the genomic resolution to 20 kb. FIG. 3B) Graphical representation of Matryoshka design for the TwoChrs library. The first 25 steps divide the region into larger intervals of 500 kb and the last 5 steps further subdivide each Mainstreet step to bring the genomic resolution to 100 kb. FIG. 3C) Image of all 25 steps of the EGFR library. FIG. 3D) Image of all 30 steps of the TwoChrs library. Each sphere in FIG. 3C & DIF. 3D represents a single blink. Pseudo-colors represent rounds of imaging.

Assigning sub-intervals to their larger chromosomal segments. To identify the larger interval of which the smaller interval (FIG. 4A) is corresponding to, the inventors first implemented a dimensional reduction strategy (FIG. 4B). Initially, the pairwise distances were calculated for all the data points and determined an optimal radius for a sphere encompassing particles likely originating from the same probes. Setting the radius at R=100 nm, particles within this sphere were considered to emanate from the same genomic location/probe, with their center of mass representing a single particle. This approach significantly economized computational resources for subsequent analyses. Following dimensionality reduction, the inventors employed graph theory algorithms to predict the most probable walks between genomic loci. Utilizing Dijkstra's algorithm²⁵, directed graphs were constructed (FIG. 4C) where each center-of-mass data point served as a node. A Dijkstra's algorithm was selected as it is highly effective for finding the shortest path in graphs with non-negative weights and leverages a priority queue to selectively process the nearest unvisited vertex, optimizing performance particularly well for graphs represented with adjacency lists. Edge weights were assigned based on Euclidean distances between nodes and their corresponding measurement precision, establishing connections between nodes with the highest precision.

Upon confident delineation of paths for individual larger segments, pairwise distances between each sub-segment particle and all edges across the graphs were computed. The larger segment affiliation of each sub-segment particle was predicted by selecting the nearest sub-segments particle-graph edge (FIG. 4D).

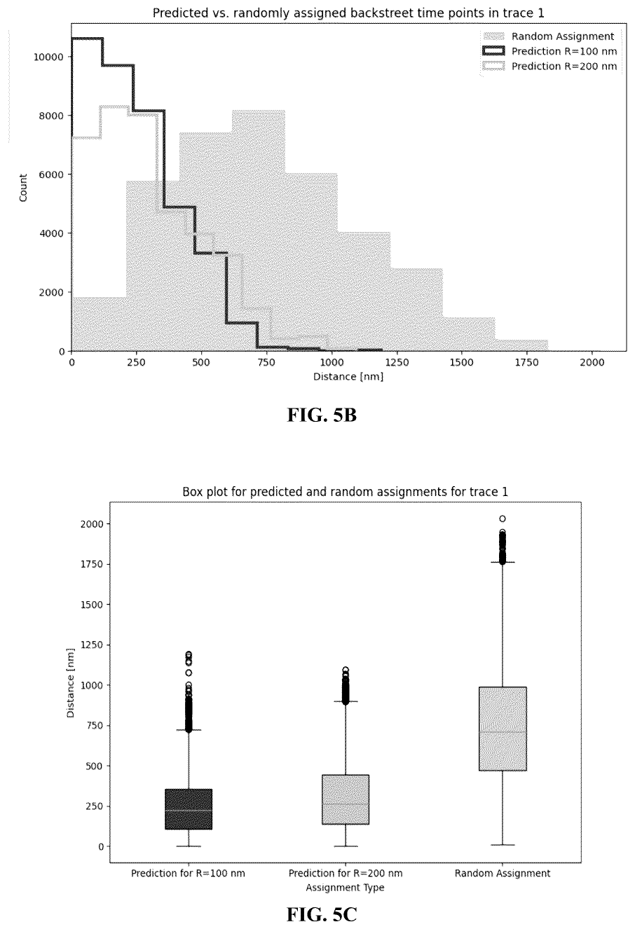

To validate the robustness of the algorithm, the inventors compared performance of the algorithm against randomly assigning particles originating from sub-intervals to larger intervals, ensuring its efficacy and reliability. A p-value very close to 0 for the null hypothesis was obtained, which shows that the algorithm was no better than random assignments, with the alternative hypothesis indicating that the algorithm works significantly better than random assignments. This statistical analysis demonstrates the algorithm's performance and its superiority over random assignment methods (FIG. 5A TO 5C). FIG. 5A shows the fraction of BS assignments on each MS steps. FIG. 5B shows the distributions for pairwise distances between the BS particles and their edge assignments on the graph for R=100 nm, R=200 nm, and random assignments. The performance of the algorithm matching BS particles to an edge on the graph is significantly better than randomly choosing them by comparing the spatial approximation of the match pairs. FIG. 5C shows all cases in box plots and validates the better performance.

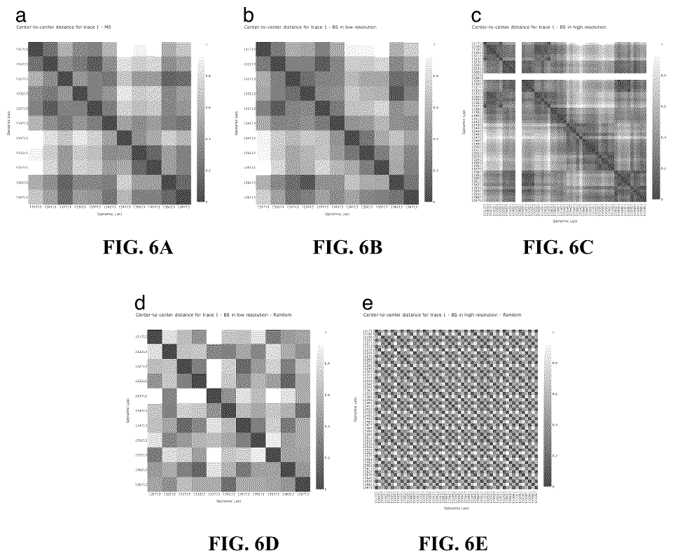

The performance of the pipeline is also validated by plotting the center-to-center contact maps between time points obtained with mainstreet localizations (FIG. 6A), with backstreet localizations after finding their mainstreet matches both in the same binning as mainstreets and in finer binning for higher resolution (FIG. 6B, 6C), and with random assignments of backstreets both in the same binning as mainstreets and in finer binning for higher resolution (FIG. 6D, 6E). Plotting backstreet localization contact maps with the same binning as mainstreet maps shows that, after assigning backstreets with the pipeline, at low resolutions, a similar pairwise distance pattern was obtain as observed with mainstreet localizations, which directly stems from the empirical data. Contact maps obtained with random assignments demonstrate that the pipeline predicts backstreet locations more accurately than randomization. Thus, Matryoshka reveals genome folding at finer scales.

Library design. The EGFR library was designed to cover a 2 Mb region (roughly 1 Mb before and after the EGFR gene locus), specifically, chr7: 54,100,001-56,100,000 using the novel “Matryoshka” design. To that end, the 2 Mb region to be imaged is first divided into twenty segments of 100 kb length each, with each of these being assigned a “mainstreet” name (Main 1-Main20). Each of the 100 kb segments is then further subdivided into five 20 kb sub-segments, which are assigned a “backstreet” name (Back1-Back5). These five backstreet barcodes are the same for all mainstreets and enable imaging sub-intervals of different larger intervals simultaneously. For example, the first sub-interval, which corresponds to ‘Back1’ is imaged for all of the twenty mainstreets (larger segments) simultaneously. Each oligo is designed to have a unique genome homology region, flanked by a mainstreet and a backstreet barcode, and these are flanked with a universal pair of primers that allows amplifying the entire oligo library at once (FIG. 1). Genome homology regions were downloaded from PaintSHOP26 using the pre-mined hg38 probe library and default parameters (no repeats, 200 max off-target score, 5 max K-mer count, and minimum probe value 0). The downloaded sequences were appended with the OligoLego³ MATLAB script (github.com/gnir/OligoLego), which appends the target-specific barcodes and universal PCR primers to either end of the genome homology regions.

The TwoChrs library was designed to cover a 7.7 Mb region corresponding to chr9: 130,700,000-138,400,000 and a 4.31 Mb region, which is, chr22: 19,000,001-23,318,037. The library is first divided into 25 targets of 500 kb length each (9 targets in chr22 and 16 targets in chr9), then subdivided into five sub-segments of 100 kb each, following the same Matryoshka principles described for the EGFR library. Genome homology region sequences were downloaded from PaintSHOP and appended with OligoLego, in the same way described for the EGFR library.

Library Amplification. Oligopaint libraries were ordered from Twist Bioscience (www.twistbioscience.com), whereas the primer, bridge, and toehold sequences were ordered from IDT (www.idtdna.com) with standard desalting. The readout probes carrying an Alexa647N fluor at each end (two on each readout probe in total) were ordered from IDT with HPLC purification. The probe libraries were amplified with PCR as previously described²⁷. Briefly, libraries were reconstituted to 20 ng/μL and amplified via PCR (Kapa Hi-Fi PCR kit, Fisher 50-196-5217), cleaned (Zymo D4033/D4029), and eluted with ultra pure water. The dsDNA product was then in vitro transcribed into RNA at 37° C. for 16 hours (NEB HiScribe, E2040S). The RNA was then reverse transcribed into cDNA using Maxima H Minus Reverse Transcriptase (Thermo Fisher, EP0752). The remaining RNA was digested using 0.5M NaOH and 0.25M EDTA to obtain ssDNA, which was then cleaned and concentrated to a concentration of 200 pmol/μL. Product size was validated using a 2% agarose gel.

Cell culture. Human microglia (HMC3, ATCC: CRL-3304) were cultured at 37° C. and 5% CO₂ in Eagle's Minimum Essential Media (EMEM, ATCC: 30-2003) supplemented with 10% (v/v) Fetal Bovine Serum (FBS). IMR-90 (ATCC: CCL-186) were cultured at 37° C. and 5% CO₂ in Minimum Essential Medium (MEM) Alpha (1×)+GlutaMAX (Gibco: 32561-037) supplemented with 10% ((v/v) Fetal Bovine Serum (FBS) and 5% (v/v) Penicillin/Streptomycin. GM12878 (Coriell Institute: GM12878) were cultured at 37° C. and 5% CO₂ in Roswell Park Memorial institute 1640 media (RPMI 1640, Gibco: 11875-093) supplemented with 15% (v/v) Fetal Bovine Serum (FBS) and 5% (v/v) Penicillin/Streptomycin.

Sample preparation. Adherent cells were lifted with TripLE (Thermo Fisher, 12605010), diluted to concentrations suitable for each cell type (5×10⁵ cells per mL for HMC3, 1×10⁶ cells per mL for IMR90), and 150 μL of cell suspension was added to Ibidi single channel μ-Slides I 0.2 (Ibidi, 80167). Slides were left at 37° C. and 5% CO₂ overnight to allow cell attachment. The following day, cell media was removed, cells were washed once with PBS and then fixed in 4% paraformaldehyde (16% PFA diluted to 4% with PBS, Electron Microscopy Services, 15710) solution for 10 minutes at room temperature. The fixed cells were washed and either used immediately or stored in PBS at 4° C. for up to two weeks. On the day of use, samples were washed with PBS followed by permeabilization of the membrane using 0.5% Triton-X (Sigma Aldrich, 93443) for 10 minutes at room temperature. From here, all subsequent incubations were done at room temperature on a shaker unless otherwise specified. Samples were washed with 1×PBST (1×PBS+0.1% (v/v) Tween-20) for 2 minutes, 0.1N HCl for 5 minutes, twice with 2×SSCT (2×SSC+0.1% (v/v) Tween-20) for 1 minute, and last with 2×SSCT+50% Formamide (v/v) for 2 minutes. The sample was then incubated with fresh 2×SSCT+50% Formamide for 20 minutes on a heat block placed in a 60° C. water bath. Channel was then dried completely, and liquid was quickly replaced with a hybridization solution containing primary library probes (50% Formamide+2×SSCT+10% Dextran Sulfate (w/v)+400 ng/μL RNase A, +4 pmol/μL library). The sample containing primary hybridization solution was then denatured for 3 minutes on a heat block in an 80° C. water bath. Sample with primary hybridization was left in a humidity chamber overnight at 42° C. The following day, primary hybridization solution was removed, and the sample was washed with 2×SSCT, followed by 4 washes of 5 minutes each on a heat block in a 60° C. water bath using SSCT prewarmed to 60° C. After the warm washes, the sample was washed twice with room temperature 2×SSCT for 2 minutes each time, and then once with 1×PBS. At this point gold nano-urchins (Sigma, 797707) were sonicated for 10 minutes and then diluted 1:30 with 1×PBS. The diluted nano-urchins were added to the sample and centrifuged at 500 G for 3 minutes. Secondary hybridization solutions (2×SSCT+0.3% Tween-20 (v/v)+30% Formamide (v/v)+500 nM bridge oligo+500 nM Alexa647 readout probe+500 nM universal readout probe+500 nM Alexa405) containing bridge oligos complimentary to both the fluorescent oligos sequences as well as the first Mainstreet barcode was then added to the sample and incubated at room temperature in the dark for 1 hour. A universal readout probe which is complementary to the forward primer appended to the library of oligos and therefore binds to the entire library is added in the first sequential step only to be used to take a reference image and setting of Z-stacks. From the second step of imaging onwards this oligo is replaced with 500 nM tocholds of the previous step to remove previous bridge sequences. Following secondary hybridizations, the sample is washed 4 times for 5 minutes each with a wash buffer (2×SSCT+35% Formamide (v/v)). Image buffer (10% (w/v) glucose, 2×SSC, 50 mM Tris, 1% (v/v) β-mercaptoethanol, and 2% (v/v) of a GLOX stock solution consisting of 100 mg/mL glucose oxidase (Sigma G2133-250KU), 7.5 mg/mL catalase (Sigma C40-500 MG), 30 mM Tris, and 30 mM NaCl) is then added to the sample for imaging.

Imaging. Imaging was done using a Bruker Vutara VXL Super Resolution Microscope connected to a Fluidgent Bruker Box VI fluidics machine. This system is run on a PC (specs) using Bruker software called SRX.

Image analysis. subsections: 1. localization algorithm and parameters (see plos genetics 2018). 2. filtering localizations (e.g., axial precision set at 100 nm). 3. Drift-correction. 4. DBSCAN. 5. Matryoshka overlap analysis. 6. Structural analysis.

Edge weight calculation. Edge weights of the graphs were assigned based on Euclidean distances between nodes and their corresponding measurement precision, establishing connections between nodes with the highest precision. Edge weights between nodes a and B are calculated using the formula:

$${weight}_{αβ} = \frac{1}{{precision}_{i} \times {distance}_{αβ}} ,$$

where precisionᵢ, calculated from the Fisher information matrix, sums over indices i=x, y, z (using Einstein summation convention) and represents the standard deviation of the parameters x, y, and z positions of the fluorophore²⁸⁻³⁰. The term distanceag is the Euclidean distance between the two nodes. According to this formula, the greater the estimation precision (the lower the precisionᵢ) of the data points and the closer their proximity, the higher the probability of a walk occurring between them.

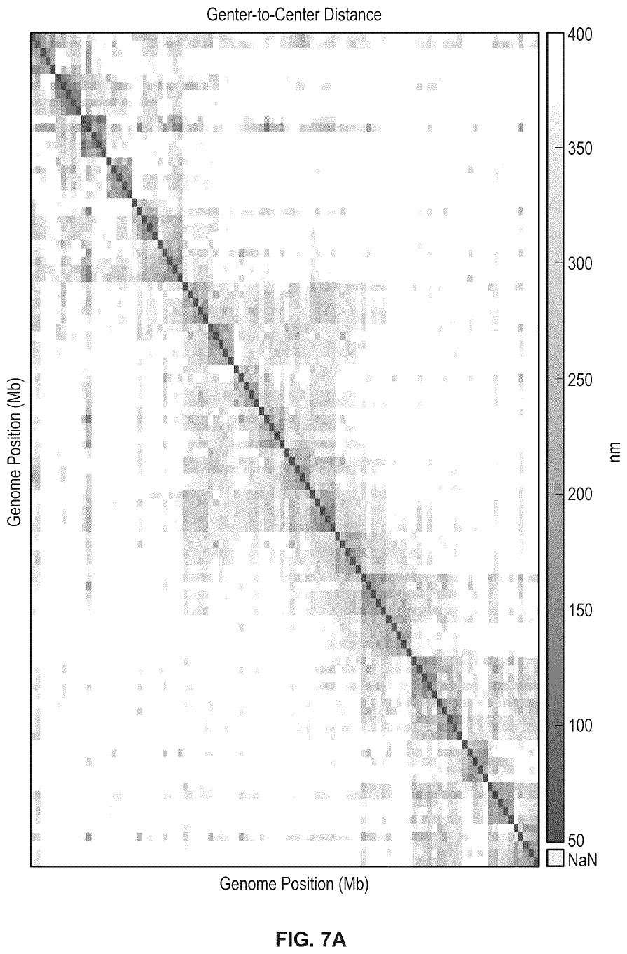

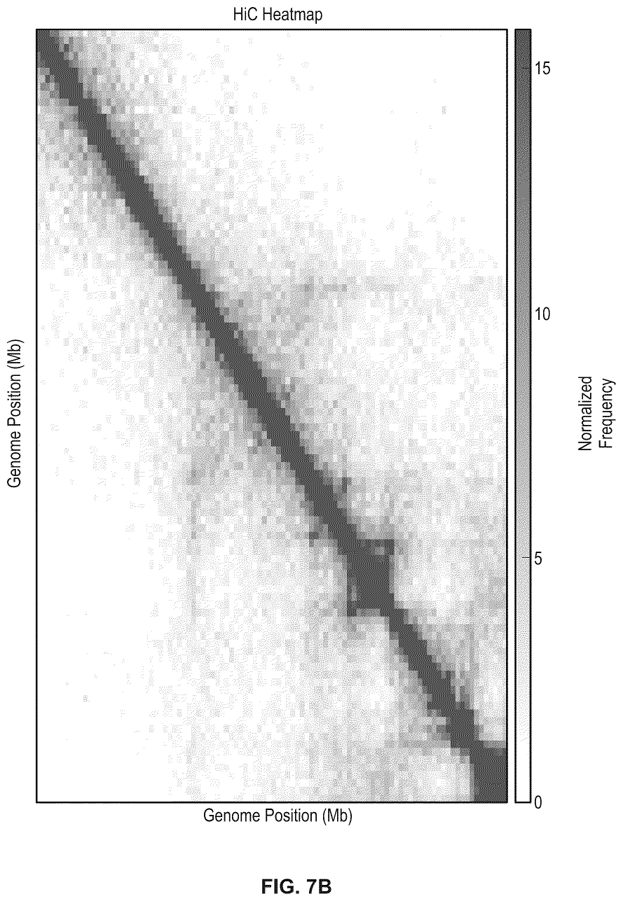

FIGS. 7A and 7B show the results from using Matryoshka. FIG. 7A shows the the mean center-to-center distance matrix, compared to FIG. 7B using Hi-C, which is a well-known method for 3D genome organization at the ensemble level. The graphs are at 20 kb, using Matryoshka from 100 kb to 20 kb.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Bintu, B. et al. Super-resolution chromatin tracing reveals domains and cooperative interactions in single cells. Science 362 (2018).
2. Wang, S. et al. Spatial organization of chromatin domains and compartments in single chromosomes. Science 353, 598-602 (2016).
3. Nir, G. et al. Walking along chromosomes with super-resolution imaging, contact maps, and integrative modeling. PLOS genetics 14, e1007872 (2018).
4. Yodai, T. et al. High-resolution spatial multi-omics reveals cell-type specific nuclear compartments. bioRxiv, 2023.2005.2007.539762 (2023).
5. Sawh, A. N. et al. Lamina-Dependent Stretching and Unconventional Chromosome Compartments in Early C. elegans Embryos. Mol Cell 78, 96-111.e116 (2020).
6. Payne, A. C. et al. In situ genome sequencing resolves DNA sequence and structure in intact biological samples. Science 371, eaay3446 (2021).
7. Hafner, A. et al. Loop stacking organizes genome folding from TADs to chromosomes. Molecular Cell 83, 1377-1392.e1376 (2023).
8. Cardozo Gizzi, A. M. et al. Microscopy-Based Chromosome Conformation Capture Enables Simultaneous Visualization of Genome Organization and Transcription in Intact Organisms. Molecular Cell 74, 212-222.e215 (2019).
9. Su, J.-H., Zheng, P., Kinrot, S. S., Bintu, B. & Zhuang, X. Genome-Scale Imaging of the 3D Organization and Transcriptional Activity of Chromatin. Cell (2020).
10. Mateo, L. J. et al. Visualizing DNA folding and RNA in embryos at single-cell resolution. Nature 568, 49-54 (2019).
11. Liu, Z. et al. Linking genome structures to functions by simultaneous single-cell Hi-C and RNA-seq. Science 380, 1070-1076 (2023).
12. Zhang, R., Zhou, T. & Ma, J. Multiscale and integrative single-cell Hi-C analysis with Higashi. Nature Biotechnology 40, 254-261 (2022).
13. Nagano, T. et al. Single-cell Hi-C reveals cell-to-cell variability in chromosome structure. Nature 502, 59-64 (2013).
14. Galitsyna, A. A. & Gelfand, M. S. Single-cell Hi-C data analysis: safety in numbers. Briefings in Bioinformatics 22 (2021).
15. Stevens, T. J. et al. 3D structures of individual mammalian genomes studied by single-cell Hi-C. Nature 544, 59 (2017).
16. Wu, H. et al. Simultaneous single-cell three-dimensional genome and gene expression profiling uncovers dynamic enhancer connectivity underlying olfactory receptor choice. Nature Methods (2024).
17. Flores, V., Farabella, I. & Nir, G. Genome-wide tracing to decipher nuclear organization. Current Opinion in Cell Biology 82, 102175 (2023).
18. Xie, L. & Liu, Z. Single-cell imaging of genome organization and dynamics. Molecular Systems Biology 17, e9653 (2021).
19. Kim, H. H.-S. & Lakadamyali, M. Microscopy methods to visualize nuclear organization in biomechanical studies. Current Opinion in Biomedical Engineering 30, 100528 (2024).
20. Takei, Y. et al. Integrated spatial genomics reveals global architecture of single nuclei. Nature 590, 344-350 (2021).
21. Liu, M. et al. Multiplexed imaging of nucleome architectures in single cells of mammalian tissue. Nature Communications 11, 2907 (2020).
22. Dekker, J. et al. Spatial and temporal organization of the genome: Current state and future aims of the 4D nucleome project. Molecular Cell 83, 2624-2640 (2023).
23. Zhuang, X. Spatially resolved single-cell genomics and transcriptomics by imaging. Nature methods 18, 18-22 (2021).
24. Takei, Y. et al. Single-cell nuclear architecture across cell types in the mouse brain. Science 374, 586-594 (2021).
25. Dijkstra, E. W. A note on two problems in connexion with graphs. Numerische Mathematik 1, 269-271 (1959).
26. Hershberg, E. A. et al. PaintSHOP enables the interactive design of transcriptome- and genome-scale oligonucleotide FISH experiments. Nature Methods 18, 937-944 (2021).
27. Beliveau, B. J. et al. in Super-Resolution Microscopy, Vol. 1663. (ed. H. Erfle) 231-252 (Springer, 2017).
28. Ober, R. J., Ram, S. & Ward, E. S. Localization accuracy in single-molecule microscopy. Biophysical journal 86, 1185-1200 (2004).
29. Tahmasbi, A., Ward, E. S. & Ober, R. J. Determination of localization accuracy based on experimentally acquired image sets: applications to single molecule microscopy. Optics express 23, 7630-7652 (2015).
30. Huang, F. et al. Video-rate nanoscopy using sCMOS camera-specific single-molecule localization algorithms. Nature methods 10, 653-658 (2013).

What is claimed is:

1. A system of determining genome organization at high spatial and genomic resolution comprising:
a microscope for imaging one or more chromosomes fixed on a substrate;
a library comprising one or more imaging oligonucleotides capable of binding to the one or more chromosomes, each imaging oligonucleotide comprising a genome homology region sequence that binds to a genomic sequence, two or more barcode sequences, and two or more universal primer sequences;
reagents for amplification of two or more barcodes with universal primers;
wherein the microscope is capable of capturing:
one or more images of the one or more chromosomes at a first resolution by subdividing the one or more chromosome into two or more first segments having a first length;
imaging the two or more first segments at a second resolution by subdividing each of the two or more first segments into two or more second segments having a second length; and
subsequently imaging additional subsegments by subdividing each prior segment into two or more subsequent segments having two or more smaller lengths; and
a processor capable of processing the one or more images at a first, second or subsequent resolution, to determine a resolution of nested images between the lowest and highest resolutions.

2. The system of claim 1, wherein the barcode sequences are 3′, 5′, or both 3′ and 5′ from the genome homology region sequence.

3. The system of claim 1, further comprising increasing or decreasing one or more lengths of the one or more subsequent segments to increase one or more times a resolution of the genome organization at high spatial and genomic resolution.

4. The system of claim 1, wherein the barcode sequences are amplified with a universal primer and a chromophore or visualizing agent.

5. The system of claim 1, wherein the first segment has a length of an entire chromosome, the second segment subdivides the length of the first segment 2, 3, 4, 5, 6, 7, 8, 9, 10, 15, 20, 25, 30, 35, 40, 45, 50, 60, 70, 80, 90, 100, 200, 250, 300, 400, 500, 600, 700, 750, 800, 1,000, 2,000, 3,000, 4,000, 5,000, 6,000, 7,000, 8,000, 9,000, 10,000 times or more, and wherein each additional subdivision of additional subsegments is subdivided 2, 3, 4, 5, 6, 7, 8, 9, 10, 15, 20, 25, 30, 35, 40, 45, 50, 60, 70, 80, 90, 100 times or more, down to a resolution of an oligonucleotide.

6. The system of claim 1, wherein the genome organization imaged is used to determine one or more of: loop domain borders, structural differences between chromosome compartments and homologous chromosomes, how loop stacking organizes chromatin folding, and how gene activity is directly linked to chromosome structure.

7. The system of claim 1, wherein the imaging is by Single Molecule Localization Microscopy (SMLM) or Stochastic Optical Reconstruction Microscopy (STORM).

8. The system of claim 1, wherein the chromosomes are traced by ball-and-stick tracing (BST) or volumetric chromatin tracing (VCT).

9. The system of claim 1, wherein the imaging additional subsegments by subdividing each prior segment into two or more subsequent segments is selected from 1, 2, 3, 4, 5, 6, 7, 8, 9, 10, 11, 12, 13, 15, 16, 17, 18, 19, 20, 25, 30, 40, 50 or more subsequent rounds or waves of subdivisions.

10. The system of claim 1, wherein the subdivision of segments follows the formula: y=x\hat{ }n, where y is the number of total targets (imaged loci), x is the number of rounds of imaging per wave, and n is the number of waves.

11. The system of claim 1, wherein the imaging of the one or more chromosomes achieves a resolution of 8, 10, 20, 30, 40, 50, 60, 70, 80, 90, 100, 200, 250, 300, 400, 500, 750, 1,000 bp, 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 kb, or 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 Mb, or portions of a chromosome.

12. The system of claim 1, wherein the one or more chromosomes are imaged concurrently.

13. A method of determining genome organization at high spatial and genomic resolution comprising:
obtaining or having obtained one or more chromosomes;
binding to the one or more chromosomes a library comprising one or more imaging oligonucleotides, each imaging oligonucleotide comprising a genome homology region sequence that binds to a genomic sequence, two or more barcode sequences, and two or more universal primer sequences;
imaging the one or more chromosomes at a first resolution by subdividing the one or more chromosomes into two or more first segments having a first length;
imaging the two or more first segments at a second resolution by subdividing each of the two or more first segments into two or more second segments having a second length; and
subsequently imaging additional subsegments by subdividing each prior segment into two or more subsequent segments having two or more smaller lengths.

14. The method of claim 13, wherein the barcode sequences are 3′, 5′, or both 3′ and 5′ from the genome homology region sequence.

15. The method of claim 13, wherein the barcode sequences are amplified with a universal primer and a chromophore or visualizing agent.

16. The method of claim 13, further comprising increasing or decreasing one or more lengths of the one or more subsequent segments to increase one or more times a resolution of the genome organization at high spatial and genomic resolution.

17. The method of claim 13, wherein the first segment has a length of an entire chromosome, the second segment subdivides the length of the first segment 2, 3, 4, 5, 6, 7, 8, 9, 10, 15, 20, 25, 30, 35, 40, 45, 50, 60, 70, 80, 90, 100, 200, 250, 300, 400, 500, 600, 700, 750, 800, 1,000, 2,000, 3,000, 4,000, 5,000, 6,000, 7,000, 8,000, 9,000, 10,000 times or more, and wherein each additional subdivision of additional subsegments is subdivided 2, 3, 4, 5, 6, 7, 8, 9, 10, 15, 20, 25, 30, 35, 40, 45, 50, 60, 70, 80, 90, 100 times or more, down to a resolution of an oligonucleotide.

18. The method of claim 13, wherein the genome organization imaged is used to determine one or more of the following: loop domain borders, structural differences between chromosome compartments and homologous chromosomes, how loop stacking organizes chromatin folding, and how gene activity is directly linked to chromosome structure.

19. The method of claim 13, wherein the imaging is by Single Molecule Localization Microscopy (SMLM) or Stochastic Optical Reconstruction Microscopy (STORM).

20. The method of claim 13, wherein the chromosomes are traced by ball-and-stick tracing (BST) or volumetric chromatin tracing (VCT).

21. The method of claim 13, wherein the imaging additional subsegments by subdividing each prior segment into two or more subsequent segments is selected from 1, 2, 3, 4, 5, 6, 7, 8, 9, 10, 11, 12, 13, 15, 16, 17, 18, 19, 20, 25, 30, 40, 50 or more subsequent rounds or waves of subdivisions.

22. The method of claim 13, wherein the subdivision of segments follows the formula: y=x\hat{ }n, where y is the number of total targets (imaged loci), x is the number of rounds of imaging per wave, and n is the number of waves.

23. The method of claim 13, wherein the imaging of the one or more achieves a resolution of 8, 10, 20, 30, 40, 50, 60, 70, 80, 90, 100, 200, 250, 300, 400, 500, 750, 1,000 bp, 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 kb, or 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 Mb, or portions of a chromosome.

24. The method of claim 13, wherein one or more chromosomes are imaged concurrently.

25. A kit comprising in one or more vials:
one or more reagents for fixing one or more chromosomes;
a library capable of binding to the one or more chromosomes comprising one or more imaging oligonucleotides, wherein each imaging oligonucleotide comprising a genome homology region sequence that binds to a genomic sequence, two or more barcode sequences, and two or more universal primer sequences;
one or more universal oligonucleotides;
one or more amplification reagents for amplification products from the two or more barcode sequences;
one or more imaging reagents for imaging products from the two or more barcode sequences; and
instructions for use of the kit to image and determine a genome organization at high spatial and genomic resolution.

26. The kit of claim 25, wherein the barcode sequences are 3′, 5′, or both 3′ and 5′ from the genome homology region sequence.

27. The kit of claim 25, wherein the barcode sequences are amplified with a universal primer and a chromophore or visualizing agent.

28. The kit of claim 25, wherein the first segment has a length of an entire chromosome, the second segment subdivides the length of the first segment 2, 3, 4, 5, 6, 7, 8, 9, 10, 15, 20, 25, 30, 35, 40, 45, 50, 60, 70, 80, 90, 100, 200, 250, 300, 400, 500, 600, 700, 750, 800, 1,000, 2,000, 3,000, 4,000, 5,000, 6,000, 7,000, 8,000, 9,000, 10,000 times or more, and wherein each additional subdivision of additional subsegments is subdivided 2, 3, 4, 5, 6, 7, 8, 9, 10, 15, 20, 25, 30, 35, 40, 45, 50, 60, 70, 80, 90, 100 times or more, down to a resolution of an oligonucleotide.

29. The kit of claim 25, wherein the genome organization imaged is used to determine one or more of: loop domain borders, structural differences between chromosome compartments and homologous chromosomes, how loop stacking organizes chromatin folding, and how gene activity is directly linked to chromosome structure.

30. The kit of claim 25, wherein the imaging is by Single Molecule Localization Microscopy (SMLM) or Stochastic Optical Reconstruction Microscopy (STORM).

31. The kit of claim 25, wherein the one or more chromosomes are traced by ball-and-stick tracing (BST) or volumetric chromatin tracing (VCT).

32. The kit of claim 25, wherein imaging additional subsegments by subdividing each prior segment into two or more subsequent segments is selected from 1, 2, 3, 4, 5, 6, 7, 8, 9, 10, 11, 12, 13, 15, 16, 17, 18, 19, 20, 25, 30, 40, 50 or more subsequent rounds or waves of subdivisions.

33. The kit of claim 25, wherein the subdivision of segments follows the formula: y=x\hat{ }n, where y is the number of total targets (imaged loci), x is the number of rounds of imaging per wave, and n is the number of waves.

34. The kit of claim 25, wherein imaging of the one or more chromosomes achieves a resolution of 8, 10, 20, 30, 40, 50, 60, 70, 80, 90, 100, 200, 250, 300, 400, 500, 750, 1,000 bp, 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 kb, or 1.25, 1.5, 1.75, 2, 2.5, 3, 4, 5, 6, 7, 7.5, 8, 9, 10, 20, 25, 30, 40, 50, 60, 70, 75, 80, 90, 100, 200, 250, 300, 400, 500, 750, 1,000 Mb, or portions of a chromosome.

35. The kit of claim 25, wherein the one or more chromosomes are imaged concurrently.
